(19) United States
(12) Patent Application Publication
(10) Pub. No.: US 2010/0259395 A1
(43) Pub. Date: Oct. 14, 2010

(54) PATIENT MONITORING SYSTEM AND METHOD

(75) Inventor: Sridhar Nuthi, Sussex, WI (US)

Correspondence Address: PETER VOGEL;GE HEALTHCARE, 20225 WATER TOWER BLVD., MAIL STOP W492, BROOKFIELD, WI, 53045, US

(73) Assignee: GENERAL ELECTRIC COMPANY, Schenectady, WI (US)

(21) Appl. No.: 12/420,533

(22) Filed: Apr. 8, 2009

(51) Int. Cl. G08B 23/00; A61B 5/00

(52) U.S. Cl. 340/573.1; 600/300

(57) ABSTRACT

A system and associated method include a patient monitoring system configured to obtain patient data from a plurality of monitored patients, an alarm, and a processor. The processor is coupled with the patient monitoring system and the alarm. The processor is configured to generate a custom alarm trigger for each of the monitored patients, and to selectively actuate the alarm based on an analysis of the custom alarm triggers and the patient data.

### BACKGROUND OF THE INVENTION

The subject matter disclosed herein relates to a patient monitoring system and method.

Patient monitoring systems enable a single technician to monitor one or more patients. The patient monitoring systems may, for example, comprise an electrocardiograph, a blood pressure monitor, a thermometer, and/or a pulse oximeter in order to monitor several different physical characteristics of each patient. The patient monitoring systems typically include an audible and/or visible alarm adapted to alert hospital personnel if of one or more selectable criteria are met.

One problem with conventional patient monitoring systems is the process of selecting the alarm criteria can produce false alarms. False alarms unnecessarily tax hospital resources and interfere with the working environment.

### BRIEF DESCRIPTION OF THE INVENTION

The above-mentioned shortcomings, disadvantages and problems are addressed herein which will be understood by reading and understanding the following specification.

In an embodiment, a system includes a patient monitoring system configured to obtain patient data from a plurality of monitored patients, an alarm, and a processor. The processor is coupled with the patient monitoring system and the alarm. The processor is configured to generate a custom alarm trigger for each of the monitored patients, and to selectively actuate the alarm based on an analysis of the custom alarm triggers and the patient data.

In another embodiment, a method includes generating a custom alarm trigger for each of a plurality of monitored patients, obtaining patient data for each of the plurality of monitored patients, and selectively actuating an alarm based on an analysis of the custom alarm triggers and the patient data.

In another embodiment, a method includes obtaining a medical history for each of a plurality of monitored patients, and generating a custom alarm trigger for each of the plurality of monitored patients. Each of the custom alarm triggers are based on one of the medical histories, and each of the custom alarm triggers include a triggering criteria. The method also includes obtaining patient data for each of the monitored patients, and actuating an alarm only if one of the triggering criteria is met by the patient data.

Various other features, objects, and advantages of the invention will be made apparent to those skilled in the art from the accompanying drawings and detailed description thereof.

### BRIEF DESCRIPTION OF THE DRAWINGS

FIG. 1 is a schematic representation of a centralized patient monitoring system in accordance with an embodiment; and

FIG. 2 is a flow chart illustrating a method in accordance with an embodiment.

### DETAILED DESCRIPTION OF THE INVENTION

In the following detailed description, reference is made to the accompanying drawings that form a part hereof, and in which is shown by way of illustration specific embodiments that may be practiced. These embodiments are described in sufficient detail to enable those skilled in the art to practice the embodiments, and it is to be understood that other embodiments may be utilized and that logical, mechanical, electrical and other changes may be made without departing from the scope of the embodiments. The following detailed description is, therefore, not to be taken as limiting the scope of the invention.

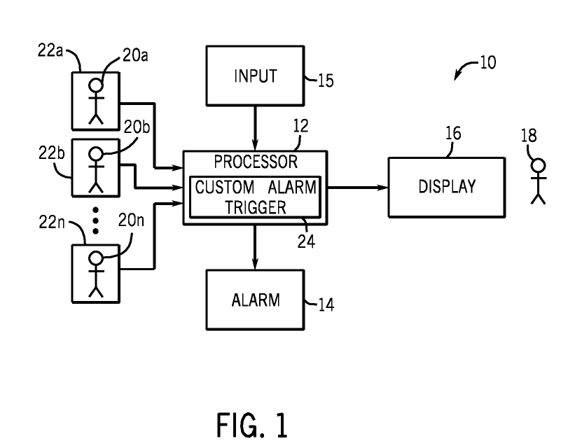

Referring to FIG. 1, a centralized patient monitoring system 10 is shown in accordance with one embodiment. The centralized patient monitoring system 10 includes a processor 12, an alarm 14, an input device 15 and a display 16 that are adapted to enable a single technician 18 to generally simultaneously monitor a plurality of patients 20a-20n. While shown and described as components of the centralized patient monitoring system 10, it should be appreciated that the processor 12, the alarm 14, the input device 15 and/or the display 16 may be implemented with other patient monitoring systems such as, for example, a single patient bedside monitoring system.

The processor 12 is coupled with a plurality of discrete patient monitoring systems 22a-22n such as, for example, via a conductor or a wireless connection. The discrete patient monitoring systems 22a-22n are each configured to monitor one of the patients 20a-20n, to generate patient data based on the specific characteristics being monitored, and to transmit the patient data to the processor 12. In a non-limiting manner, the discrete patient monitoring systems 22a-22n may comprise an electrocardiograph, a blood pressure monitor, a thermometer, and/or a pulse oximeter. Correspondingly, the patient data generated by the patient monitoring systems 22a-22n may comprise electrocardiogram (ECG) data, blood pressure data, temperature data, and/or pulse data.

The processor 12 is configured to generate a custom alarm trigger 24 as described in more detail hereinafter, and to selectively actuate or sound the alarm 14 based on the custom alarm trigger 24 and the patient data from the discrete monitoring systems 22a-22n. The alarm 14 may comprise an audible device (e.g., a loudspeaker) and/or a visual device (e.g., a flashing light).

The custom alarm trigger 24 defines the triggering criteria that must be met in order to sound the alarm 14. For purposes of this disclosure, a “custom” alarm trigger is an alarm trigger that is customized based on the needs of a specific patient and/or is generated based on information pertaining to a specific patient. Accordingly, each of the patients 20a-20n may have a custom alarm trigger defining unique triggering criteria that are specifically appropriate to their individual needs. It should be appreciated that, by defining a custom alarm trigger in the manner described herein, the potential for false alarms is reduced as compared to a system in which a common alarm trigger is applied to every patient.

The input device 15 is coupled with the processor 12 such as, for example, via a conductor or a wireless connection. In a non-limiting manner, the input device 15 may include a keyboard, mouse, joystick, touch pad, etc. The input device 15 is configured to facilitate the manual transmission of data to the processor 12.

The display 16 is coupled with the processor 12 such as, for example, via a conductor or a wireless connection. The display 16 is configured to visually convey the patient data from the discrete monitoring systems 22a-22n such that each of the patients 20a-20n can be generally simultaneously monitored by the technician 18. While depicted as a single device, it should be appreciated that the display 16 may comprise multiple displays. Alternatively, the display 16 may comprise a single display divided into multiple partitions.

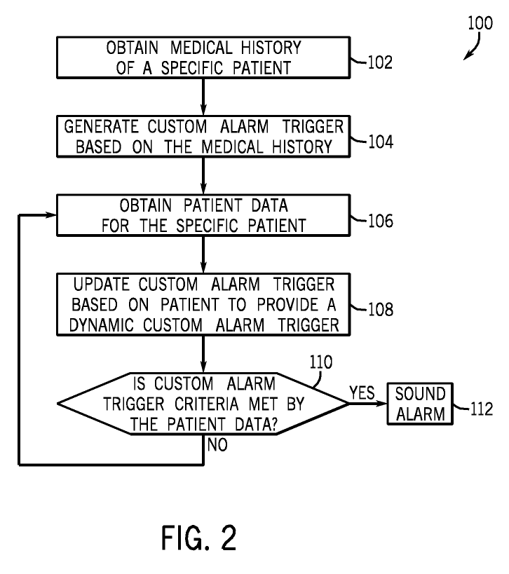

Referring to FIG. 2, a method 100 will now be described in accordance with an embodiment. The method 100 comprises a plurality of steps 102-112. One or more of the steps 102-112 may be performed by the processor 12 (shown in FIG. 1). The technical effect of the method 100 is to facilitate the process of monitoring one or more patients in a manner adapted to reduce false alarms.

Referring now to FIGS. 1 and 2, at step 102 the method 100 obtains a medical history of a specific patient (e.g., one of the patients 20a-20n). In a non-limiting manner, the medical history could include any previous diagnosis or medical conditions, medications, allergies, symptoms, treatments, etc. The medical history may also include more general information pertaining to the patient's age, sex, weight, fitness level, etc. The medical history may be obtained at step 102 by manually downloading the relevant information via input device 15 onto the processor 12 when the patient is admitted to the hospital.

According to an embodiment, the medical history obtained at step 102 may be retained and compiled in the form of a database. As the method 100 is implemented on more and more patients, the associated number of medical histories can become large enough to generate a health index. The health index may comprise an alphanumeric character adapted to classify a given patient based on criteria such as race, age, weight, preconditions, and current health. In this manner, a newly admitted patient can be assigned a health index based on similarities with any of the previous patients included in the database. The health index may then be implemented to profile the newly admitted patient for purposes of generating a custom alarm trigger (as described in more detail hereinafter), diagnosing the patient, and/or to treating the patient.

At step 104, the method 100 generates the custom alarm trigger 24 based on the medical history from step 102. As previously indicated, the custom alarm trigger 24 defines the triggering criteria that must be met in order to sound the alarm 14. In general terms, the custom alarm trigger 24 may comprise a patient data range or limit specifying the conditions under which the alarm 14 is to be triggered. According to another embodiment, the custom alarm trigger 24 may comprise minimum or maximum allowable rates of change applicable to specific types of monitored patient data that define the conditions under which the alarm 14 is to be triggered. According to yet another embodiment, the custom alarm trigger 24 may comprise trends, patterns, or morphologies defined by the monitored patient data that specify the conditions under which the alarm 14 is to be triggered. The following will provide several more specific non-limiting examples in which a custom alarm trigger can be generated based on a given patient's medical history.

Assume for purposes of a first example that an 80-year-old patient is admitted with symptoms including severe chest pains. Assume further that the patient has been previously diagnosed with cardiomyopathy. The relevant medical history to be obtained at step 102 may include the patient's age, symptoms, and previous diagnosis. Based on this medical history, it may be decided at step 104 to generate a custom alarm trigger focusing exclusively or predominantly on monitored patient data that is correlated with cardiac instability (e.g., heart rate, blood pressure, ECG data). Therefore, by generating a more focused custom alarm trigger, issues unrelated to the patient's cardiac activity would be less likely to produce a false alarm.

Assume for purposes of a second example that a 50-year-old overweight patient in poor physical condition is admitted. The relevant medical history to be obtained at step 102 may include the patient's age, weight and level of physical fitness. Based on this medical history, it may be decided at step 104 to generate a custom alarm trigger with more tolerant patient data triggering criteria (e.g., broader patient data range and/or higher allowable rate of change). The more tolerant patient data triggering criteria may be justifiable based on the assumption that any physical exertion by the patient of the present example will likely cause any measured patient data (e.g., vital signs) to vary by a greater amount as compared with a more athletic patient. Therefore, by generating a more tolerant custom alarm trigger, normal physical activity by the patient of the present example would be less likely to produce a false alarm.

At step 106, the method 100 obtains patient data from the selected patient (e.g., one of the patients 20a-20n). This step may, for example, be performed by acquiring the patient data with a patient monitoring system (e.g. one of the discrete patient monitoring systems 22a-22n), and transferring the acquired patient data to the processor 12. According to another example, the patient data may comprise a diagnosis, care plan and/or assessment from a physician that is manually input via input device 15 into the processor 12.

At step 108, the method 100 updates the custom alarm trigger (from step 104) based on the patient data (from step 106) to provide a dynamic custom alarm trigger. For purposes of this disclosure, a “dynamic” alarm trigger is an alarm trigger that can be updated or revised in light of new information (e.g., patient data from one of the discrete patient monitoring systems 22a-22n). The following will provide several non-limiting examples in which a custom alarm trigger can be updated based on patient data.

As one example, a patient's current state of health as determined by a physician may be relied upon to update the custom alarm trigger. If a given patient's health is deteriorating, the custom alarm trigger may be updated to become more stringent in order to provide additional supervision. Conversely, if a given patient's health is improving, the custom alarm trigger may be updated to become more tolerant and/or the focus of the custom alarm trigger may be revised to reduce the incidence of false alarms.

As another example, a trend in the patient data may be relied upon to update the custom alarm trigger. If a given patient has received a medication adapted to address a medical condition (e.g., high blood pressure), and subsequently acquired patient data shows a resultant improvement, the custom alarm trigger may be updated to become more tolerant and/or the focus of the custom alarm trigger may be revised to reduce the incidence of false alarms.

At step 110, the method 100 is adapted to selectively actuate the alarm 14 based on an analysis of the patient data and either the custom alarm trigger from step 104 or the dynamic custom alarm trigger from step 108. According to one embodiment, at step 110 the method 100 determines if the custom alarm trigger criteria have been met by the patient data. If at step 110 it is determined that the custom alarm criteria have been met, the method 100 proceeds to step 112 at which the alarm 14 is sounded or otherwise actuated. If at step 110 it is determined that the custom alarm criteria have not been met, the method 100 returns to step 106. By returning to step 106 in the manner described, steps 106-110 can be iteratively performed in a manner that constantly updates the custom alarm trigger based on the most recent information, and monitors the condition of the patient based on the updated custom alarm trigger criteria until it becomes necessary to actuate the alarm 14.

The process of determining whether or not the custom alarm trigger criteria have been met by the patient data at step 110 may vary based on the type of alarm criteria selected to best meet the needs of a particular patient. If, for example, the custom alarm trigger comprises a permissible range for a given patient's blood pressure, the process defined at step 110 may comprise a determination of whether or not the appropriate patient's measured blood pressure values fall outside the established permissible range. Similarly, if the custom alarm trigger comprises a predetermined maximum allowable rate of change for a given patient's heart rate, the process defined at step 110 may comprise a determination of whether or not the appropriate patient's measured heart rate increases or decreases by an amount exceeding the predetermined maximum allowable rate.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

I claim:

1. A system comprising:
a patient monitoring system configured to obtain patient data from a plurality of monitored patients;
an alarm; and
a processor coupled with the patient monitoring system and the alarm, said processor configured to generate a custom alarm trigger for each of the plurality of monitored patients, and to selectively actuate the alarm based on an analysis of the custom alarm triggers and the patient data.

2. The system of claim 1, wherein the processor is configured to generate the custom alarm triggers based on a medical history of the plurality of monitored patients.

3. The system of claim 1, wherein one or more of the custom alarm triggers comprises a dynamic custom alarm trigger.

4. The system of claim 1, wherein one or more of the custom alarm triggers comprises a range of patient data defining the triggering criteria.

5. The system of claim 1, wherein one or more of the custom alarm triggers comprises a rate of change for the patient data defining the triggering criteria.

6. The system of claim 1, wherein the analysis of the custom alarm triggers and the patient data comprises a determination of whether any triggering criteria of the custom alarm triggers is met by the patient data.

7. The system of claim 1, further comprising a display configured to generally simultaneously convey the patient data for one or more of the plurality of monitored patients.

8. The system of claim 1, wherein the patient monitoring system comprises an electrocardiograph, a blood pressure monitor, a thermometer, and/or a pulse oximeter.

9. A method comprising:
generating a custom alarm trigger for each of a plurality of monitored patients;
obtaining patient data for each of the plurality of monitored patients; and
selectively actuating an alarm based on an analysis of the custom alarm triggers and the patient data.

10. The method of claim 9, further comprising obtaining a medical history for each of the plurality of monitored patients, and wherein said generating a custom alarm trigger comprises generating a custom alarm trigger based on one of the medical histories.

11. The method of claim 9, further comprising updating one of the custom alarm triggers based on the patient data to produce a dynamic custom alarm trigger.

12. The method of claim 11, wherein said selectively actuating an alarm based on an analysis of the custom alarm triggers and the patient data comprises selectively actuating an alarm based on an analysis of the custom alarm triggers, the dynamic custom alarm triggers and the patient data.

13. A method comprising:
obtaining a medical history for each of a plurality of monitored patients;
generating a custom alarm trigger for each of the plurality of monitored patients, each of the custom alarm triggers being based on one of the medical histories, each of the custom alarm triggers comprising a triggering criteria;
obtaining patient data for each of the plurality of monitored patients; and
actuating an alarm only if one of the triggering criteria is met by the patient data.

14. The method of claim 13, further comprising updating one of the custom alarm triggers based on the patient data to provide a dynamic custom alarm trigger.

15. The method of claim 13, wherein the triggering criteria comprise a range of the patient data.

16. The method of claim 13, wherein the triggering criteria comprise a rate of change for the patient data.
